(19) United States
(12) Patent Application Publication
(10) Pub. No.: US 2023/0175034 A1
(43) Pub. Date: Jun. 8, 2023

(54) METHOD FOR CHARACTERISING MICRO-ORGANISMS USING TRANSMISSION IMAGING

(71) Applicant: COMMISSARIAT A L'ENERGIE ATOMIQUE ET AUX ENERGIES ALTERNATIVES, Paris (FR)

(72) Inventors: Pierre MARCOUX, Grenoble Cedex 09 (FR); Victor BIARDEAU, Grenoble Cedex 09 (FR); Mathieu DUPOY, Grenoble Cedex 09 (FR); Frederic-Xavier GAILLARD, Grenoble Cedex 09 (FR); Joel LE GALUDEC, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 17/995,371

(22) PCT Filed: Apr. 1, 2021
(86) PCT No.: PCT/EP2021/058664

§ 371 Date: Oct. 3, 2022

(30) Foreign Application Priority Data
Apr. 3, 2020 (FR) FR2003377

(51) Int. Cl. C12Q 1/04; G01N 21/3563; G01N 21/03

(52) U.S. Cl. CPC C12Q 1/04; G01N 21/3563; G01N 21/03; G01N 2021/0385

(57) ABSTRACT

Methods for characterizing micro-organisms may include (a) depositing micro-organisms on a porous medium with a first and a second surface and pores extending from the first surface to the second surface; (b) arranging the porous medium on the surface of a nutrient medium contained in a chamber, the second surface being arranged in contact with the nutrient medium; (c) moving the porous medium in relation to the chamber; (d) positioning the porous medium between an infrared light source and an image sensor, the light source being configured to emit an incident light wave in an emission wavelength; (e) illuminating micro-organisms retained on the porous medium, using the light source and acquiring an image using the image sensor, the image allowing an observation of at least one colony of micro-organisms; and (f) characterizing the colony of microorganisms from the image acquired in the illuminating (e).

### TECHNICAL FIELD

The technical field of the invention is characterization of microorganisms via an image acquired, in a transmission configuration, in the infrared.

### PRIOR ART

There are many methods allowing colonies of microorganisms, cultivated in liquid nutrient media (broths) or on agar jelly, to be characterized. A first method consists in evaluating various visual or olfactory parameters of colonies. These parameters may be the shape of the colonies, color, odor or surface state. This method may be applied by an experienced operator, individually, i.e. colony by colony.

Certain automated and more accurate methods are destructive. They require samples of the colonies to be taken from their nutrient medium. It is for example a question of techniques based on mass spectrometry, such as MALDI-TOF mass spectrometry (MALDI-TOF standing for Matrix-Assisted Laser Desorption Ionization-Time of Flight). This is an effective method. However, it requires complex equipment and must be applied individually, to each colony. In addition, it is a destructive technique. Another potentially destructive technique used is Raman spectroscopy. This is also an individual technique that employs sophisticated equipment and requires a colony-by-colony analysis.

Multispectral imaging techniques may be used. It is a question of illuminating the colonies at various wavelengths, in the visible or ultraviolet or infrared spectral domain. In the visible, for example, bacterial colonies may be characterized on the basis of diffraction patterns.

When the nutrient medium is sufficiently transparent, the latter may be placed between a light source and an image sensor, in a transmission configuration. The image sensor may be used in a lensless configuration, i.e. with no image-forming optics between the nutrient medium and the image sensor. Such a configuration allows diffraction patterns to be obtained at one or more wavelengths. These form signatures, allowing the species of microorganisms present in the colonies to be identified. Examples of application are for example described in US20160103064 or in U.S. Pat. No. 7,465,560.

When the nutrient medium is opaque, it is not envisionable to use a transmission configuration. Applications employing backscattering have been described in US20190339199 or in US20190323959. The objective is to obtain an image representative of radiation backscattered by a bacterial colony. On account of geometrical constraints associated with the backscattering, this method is mainly limited to individual characterization of colonies.

In the infrared domain, application of transmission imaging is complicated by the fact that the nutrient media, on which the microorganisms develop, are absorbent. This is especially due to their high mass fraction of water, which may be higher than 80%.

The inventors have developed an optical method for characterizing microorganisms, in the infrared domain, in a transmission configuration: the characterized microorganisms are placed between alight source and an image sensor.

### SUMMARY OF THE INVENTION

One subject of the invention is a method for characterizing microorganisms, comprising:
a) depositing microorganisms on a porous carrier, the porous carrier comprising a first face and a second face, and pores extending from the first face to the second face, the microorganisms being retained on the first face;
b) placing the porous carrier on the surface of a nutrient medium contained in a chamber, the porous carrier being placed such that the second face is placed in contact with the nutrient medium, so that the nutrient medium diffuses from the second face to the first face, through the pores;
c) moving the porous carrier with respect to the chamber;
d) positioning the porous carrier between an infrared light source and an image sensor, the light source being configured to emit an incident light wave at an emission wavelength, the porous carrier transmitting all or some of the incident light wave at the emission wavelength;
e) illuminating the microorganisms, placed on the porous carrier, with the light source and acquiring an image with the image sensor, at the emission wavelength, the image allowing at least one colony of microorganisms to be observed;
f) characterizing the colony of microorganisms on the basis of the image acquired in step e).

The nutrient medium contains nutrients propitious to the development of colonies of microorganisms. It may comprise a bactericide or a bacteriostat. It may also comprise an isotopic label, a chromogenic label, or a molecule that has a chemical bond that absorbs infrared light at a wavelength corresponding to the emission wavelength.

The characterization may be:
an identification of the species of the microorganisms forming the colony;
or a determination of the ability of the microorganisms to develop in the nutrient medium.

According to one embodiment
step e) is repeated, the microorganisms being successively illuminated at various emission wavelengths, so as to obtain as many images as there are emission wavelengths;
in step f), the characterization is performed on the basis of the images acquired in step e).

Preferably, the porous carrier transmits a least 1% of the light at the emission wavelength, or at each emission wavelength.

Step f) may comprise extracting a thumbnail from the image acquired in step e), the thumbnail being a region of interest of the acquired image corresponding to the colony of microorganisms. In step f), the thumbnail, or each thumbnail, may form an input datum of a supervised-learning algorithm, with a view to characterizing the microorganisms forming a colony.

According to one embodiment, step a) comprises seeding the porous carrier.

According to one embodiment, step a) comprises making a fluid, liable to contain microorganisms, flow through the porous carrier, from the first face to the second face, the carrier acting as a filter, so as to retain microorganisms on the first face, while permitting the fluid to flow through the pores. The fluid may be a liquid or a gas.

According to one embodiment, in step b), the nutrient medium:
contains molecules labelled with an isotope;
or is a chromogenic substrate;
or comprises molecules having a chemical bond that absorbs light at the emission wavelength;
such that the image formed in step e), or each image formed in step e), is representative of a metabolic activity of the microorganisms in contact with the nutrient medium.

According to one embodiment, in step b), the nutrient medium comprises a bactericide or a bacteriostat, an antibiotic for example, such that the image formed in step e), or each image formed in step e), is representative of a metabolic activity of the microorganisms when they are in contact with the nutrient medium.

According to one embodiment, following step a) and prior to step b), the method comprises an acquisition of an initial image of the porous carrier, such that metabolic activity may be determined via a comparison between the initial image and at least one image acquired in step e).

The thickness of the porous carrier, between the first face and the second face, is preferably smaller than 1 mm or than 500 μm. The diameter or largest diagonal of each pore may be comprised between 5 nm and 5 μm, and preferably between 20 nm and 500 nm. The porous carrier may be obtained from a material chosen from: alumina, silicon, germanium, zinc sulfide, silicon nitride, zinc selenide, a chalcogenide glass, calcium fluoride, and potassium bromide

The invention will be better understood on reading the description of examples of embodiment that are presented, in the remainder of the description, with reference to the figures listed below.

### FIGURES

FIG. 1A shows an example of a porous carrier suitable for implementing the invention.

FIG. 1B schematically shows the porous carrier shown in FIG. 1A being brought into contact with the surface of a nutrient medium.

FIG. 1C shows an apparatus allowing an image of colonies of microorganisms placed on the porous carrier to be taken in a transmission configuration.

FIG. 2A is a plot on the one hand of an absorbance spectrum of a porous carrier made of alumina, and on the other hand of the absorption spectra of various microorganisms.

FIG. 2B is an observation, by electron microscope, of one portion of one face of a porous carrier. This figure illustrates the pores formed in the porous carrier.

FIG. 2C is an observation, by electron microscope, in a cross section of a porous carrier. This figure illustrates that the pores extend between the two faces of the porous carrier.

FIG. 3 schematically shows the main steps of a method of implementation of the invention, and of variants of the method.

FIG. 4A is a photograph of a nutrient medium, on part of which a porous carrier has been deposited. This photograph shows the ability of the porous carrier to allow colonies of microorganisms to develop.

FIGS. 4B and 4C illustrate the development of microorganisms on an agar jelly and on a porous carrier deposited on an agar jelly, respectively.

FIGS. 5A, 5B, 5C, 5D and 5E are images of colonies of microorganisms obtained using an apparatus such as described with reference to FIG. 1C, at a plurality of wavelengths. FIGS. 5A, 5B, 5C, 5D and 5E correspond to various bacterial species, respectively.

FIGS. 6A to 6F illustrate one embodiment, in which the porous carrier acts as a filter for collecting microorganisms, and as a carrier for the microorganisms during their development, and as a carrier for the microorganisms during the acquisition of images.

FIGS. 7A to 7D illustrate one embodiment in which the microorganisms are successively brought into contact with an initial nutrient medium, then a second nutrient medium. According to this embodiment, the second nutrient medium comprises molecules labelled with a label, for example an isotopic label, or a chromogenic enzyme substrate.

FIG. 7E shows colonies in contact with a chromogenic substrate.

### DESCRIPTION OF PARTICULAR EMBODIMENTS

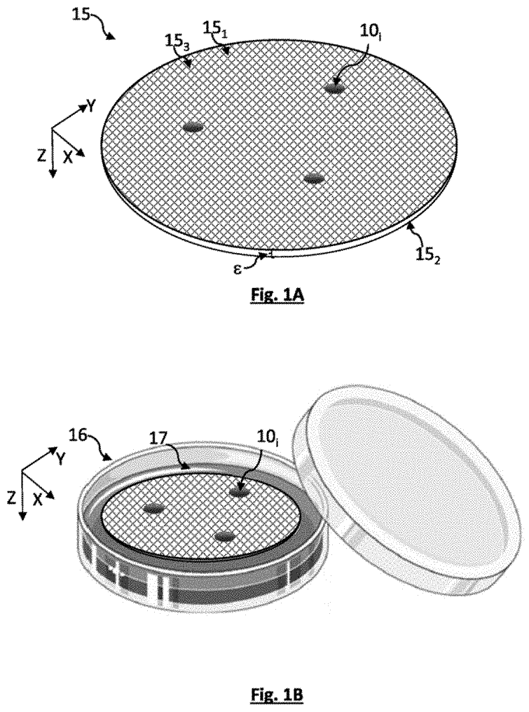

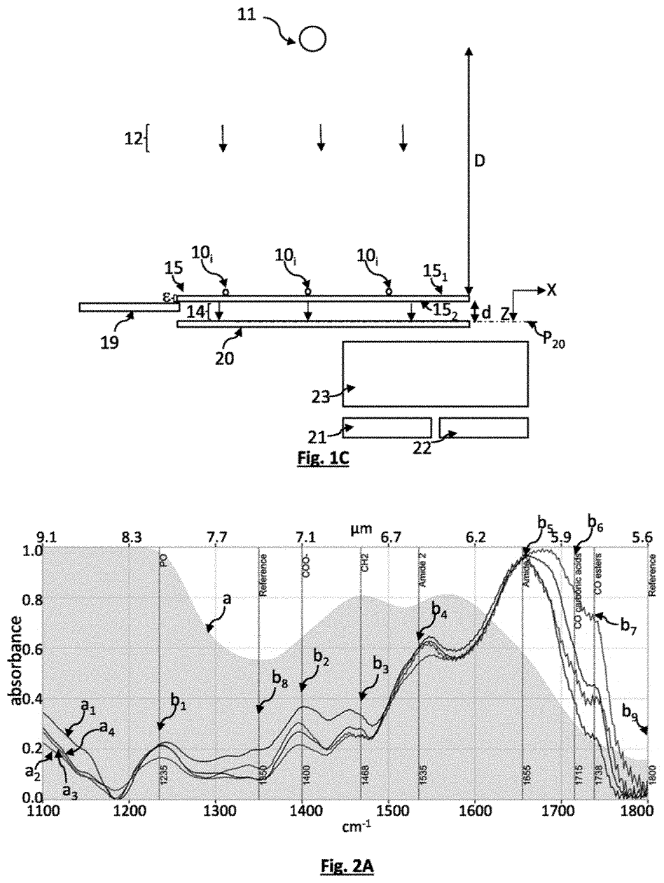

FIG. 1A schematically shows a porous carrier 15 implemented in the invention. The porous carrier 15 extends between a first face 15₁ and a second face 15₂, via a thickness e. Preferably, the first and second faces are planar, and parallel to each other. They then form two opposite faces of the carrier.

The diameter, or largest diagonal, of each face may for example be comprised between 1 cm and 10 cm, or more. The thickness ε of the porous carrier 15 is preferably smaller than 500 μm or than 1 mm or than 2 mm. It is preferably comprised between 10 μm and 1 mm, or between 10 μm and 500 μm. Depending on the thickness and on the material forming the carrier, the porous carrier 15 is flexible or rigid.

The porous carrier 15 comprises pores 15₃, extending from the first face 15₁ to the second face 15₂.

One of the functions of the porous carrier 15 is to form a carrier for microorganisms, and generally colonies of microorganisms 10ᵢ, placed on one of the faces. In the example shown, the microorganisms 10ᵢ are placed on the first face 15₁ of the carrier 15. The term microorganisms includes bacteria, archaea, microscopic fungi such as yeasts or filamentous fungi, microalgae, protozoa and parasites.

The diameter, or largest diagonal, of each pore 15₃, is preferably smaller than 5 μm, or even than 1 μm, or even than 500 nm, or even than 200 nm, and larger than 1 nm, or even than 5 nm, or even than 20 nm, or even than 50 nm. The diameter or largest diagonal is defined so as to allow microorganisms to be held on the first face 15₁ of the carrier.

FIG. 1B shows the porous carrier 15 deposited on the surface of a nutrient medium 17. The nutrient medium 17 is held in a chamber 16. The chamber 16 is for example a petri dish. The nutrient medium 17 comprises nutrients propitious to the development of colonies of microorganisms. It may for example be a question of an agar jelly, or of a liquid broth, these types of nutrient medium being known to those skilled in the art. The porous carrier 15 is deposited on a surface of the nutrient medium. The second face 15₂ makes contact with the nutrient medium 17. The nutrient medium diffuses through the pores 15₃ to the first face 15₁. Thus, the microorganisms placed on the first face 15₁ are fed by the nutrient medium 17 and may develop.

The pores 15₃ allow the nutrient medium 17 to diffuse between the two faces of the carrier 15, while retaining microorganisms on one face of the carrier. The dimension of the pores 15₃ is chosen so as to be:
large enough to allow the medium 17 to diffuse through the pores;
narrow enough to block passage of the microorganisms 10ᵢ.

Thus, the microorganisms are retained on the first face 15₁, while developing by virtue of the provision of nutrients through the pores 15₃.

FIG. 1C shows use of the carrier 15 as carrier for analyzing colonies of microorganisms 10ᵢ. The carrier 15 has been removed from the nutrient medium 17 and moved away from the chamber 16, so as to be placed facing a light source 11 and facing a detector 20. In the example shown in FIG. 1C, the carrier is placed between the light source 11 and an image sensor 20.

The light source 11 may be a light-emitting diode or a laser source. The light source 11 emits an incident light wave 12, at an emission wavelength λ. The emission wavelength λ is located in the spectral domain of the infrared. It may be a question of short wavelength infrared (SWIR), which extends between 1 and 3 μm, or of medium wavelength infrared (MWIR), which extends between 3 and 6 μm, or indeed of long wavelength infrared (LWIR), which extends between 6 and 20 μm. Thus, generally, the incident light wave 12 is emitted at a wavelength λ lying between 1 μm and 20 μm, this corresponding to a wave number comprised between 500 cm⁻¹ 10000 cm⁻¹, or between 3 μm and 20 μm. The preferred spectral range is located between 3 μm and 11 μm, which corresponds to medium wavelength infrared, and to one portion of the long wavelength infrared. Specifically, this spectral range corresponds to the domain of the wavelengths of absorption of covalent bonds commonly encountered in organic molecules.

The light source 11 is preferably a laser source. It may especially be a wavelength-tunable laser source, for example a quantum cascade laser (QCL), and in particular an external-cavity laser. The width of the emission spectral bend of the light source is preferably smaller than 10 cm⁻¹, or even than 5 cm⁻¹, or even than 1 cm⁻¹. The distance D between the light source and the carrier may be a few cm.

The light source 11 may comprise a plurality of elementary QCLs, respectively emitting in various spectral bands. The light source 11 may also be a black-body polychromatic source able to be associated with various bandpass filters defining the emission wavelength λ. Thus, the light source 11 may be polychromatic and wavelength-tunable.

The carrier 15 is placed between the light source 11 and the image sensor 20. The latter preferably lies parallel, or substantially parallel to one face of the carrier 15. The term substantially parallel means that the two elements may not be rigorously parallel, an angular tolerance of a few degrees, smaller than 20° or 10°, being acceptable. The carrier 15 may be placed on a holding element 19, configured to hold the carrier, between the light source 11 and the image sensor 20.

Under the effect of illumination by the incident light wave 12, which propagates along the propagation axis Z to the carrier 15, the latter transmits a light wave 14, which is called the transmitted light wave. The transmitted light wave 14 propagates, along the axis Z, to the image sensor 20. The carrier 15 and the microorganisms 10ᵢ may absorb one portion of the incident light wave 12. Thus, the transmitted light wave 14 corresponds to a portion of the incident light wave 12 that is not absorbed by the carrier 15 and by the colonies of microorganisms 10ᵢ.

The image sensor 20 is able to form an image of the transmitted light wave 14 in a detection plane P₂₀. In this example, the image sensor is formed by a matrix array of pixels. Each pixel is an elementary infrared detector. Each pixel may for example be a bolometer, each bolometer of the matrix array having a detection spectral band comprised between 5 μm and 20 μm. The matrix array of bolometers may for example comprise 80×80 bolometers. According to variants, the image sensor may comprise a matrix array of pyrodetectors, or of photodiodes, the detection spectral band of which is located in the infrared, and preferably in the medium wavelength infrared. Thus, each pixel of the image sensor 20 may be an elementary infrared detector
of thermal type and for example a thermoresistive or thermocapacitive or thermoelectric detector;
or a quantum photodetector and for example a photocapacitor, photoconductor, photodiode or quantum-well infrared photodetector (QWIP).

The materials from which each pixel is formed may be chosen, nonlimitingly, from lead zirconate titanate (PZT), indium antimonide (InSb), mercury cadmium telluride (HgCdTe), lead sulfide (PbS), and lead selenide (PbSe).

The distance d between the image sensor 20 and the carrier 15 is preferably smaller than 5 mm. The smaller d is, the better the spatial resolution of the image acquired by the image sensor. Thus, it is advantage for the distance d to be smaller than 1 mm, or even smaller than 500 μm.

The field of observation of the image sensor 20 is defined by the size of the sensing area of the latter. The field of observation may be larger than 1 mm², or even larger than 5 mm² or 10 mm². Thus, acquisition of a single image allows an intensity of the light wave 14 transmitted by an area of the carrier of several mm, and typically of at least 5 or 10 mm², to be obtained simultaneously. Such a configuration allows simultaneous observation of a plurality of spatially separate colonies of microorganisms.

The holding element 19 may be movable, in particular parallel to the detection plane P₂₀, so as to allow all or some of the surface of the carrier 15 to be scanned. It may in particular be a question of a motorized translation stage. The holding element 19 may also allow the carrier 15 to be rotated parallel to the detection plane.

A processing unit, for example taking the form of a microprocessor 21, is configured to perform, on the basis of the images acquired by the image sensor 20, image-interpreting operations that are described below. The microprocessor 21 is connected to a memory 22, which contains instructions relative to the image processing operations to be performed. It may also be connected to a screen 23.

One important element of the invention resides in the fact that the porous carrier 15 allows one portion of the incident light wave 12 to be transmitted to the image sensor 20. Preferably, its transmittance is higher than 0.01 (1%) or higher than 0.1 (10%). By transmittance, what is meant is a percentage of the light intensity transmitted by the material forming the porous carrier 15. It is advantageous for the transmittance to be as high as possible.

The material from which the porous carrier 15 is made may consist of or comprise a material having good transmission properties in the infrared. It may for example be a question of one of the following materials: alumina (aluminum oxide), silicon (Si), germanium (Ge), zinc sulfide (ZnS), zinc selenide (ZnSe), chalcogenide glasses based on sulfur, or on selenium or on telluride, silicon nitride (Si₃N₄), calcium fluoride (CaF₂) or potassium bromide (KBr).

Use of the porous carrier 15 allows a detector 20 to be used in a transmission mode, the carrier 15 being interposed between the light source and the detector 20. This allows individual colonies of microorganisms to be characterized using the light wave 14 transmitted by the carrier 15 and collected by the detector 20. When the detector 20 is an image sensor, sensitive to infrared, each image I_{λ} acquired by the image sensor allows a characterization of the microorganisms. More precisely, each acquired image I_{λ} is representative of an absorption of the incident light wave 12 by the microorganisms, and by the porous carrier 15, at the wavelength λ.

It will be understood that the porous carrier 15 has a dual function:
carrier of the microorganisms during their development, while in contact with a nutrient medium;
carrier of the microorganisms during an analysis via an optical method, in particular a transmission infrared imaging method.

Use of the porous carrier 15 allows the inability to form an image in transmission of microorganisms developing on nutrient media that are opaque in the infrared spectral domain to be overcome. Use of the porous carrier 15 allows the microorganisms to be moved between culture conditions, such as shown in FIG. 18, and an analyzing device, such as schematically shown in FIG. 1C. One notable advantage is that the microorganisms develop and are analyzed on the same carrier 15, transfer not being required. The morphology and structure of the colonies of microorganisms are thus preserved between the culture conditions and the analysis conditions.

Moreover, the transmission-image-based analyzing method is non-destructive. It allows, following acquisition of an image, the carrier to be once again placed on a nutrient medium. The latter may be identical to or different from an initial nutrient medium, used prior to the image acquisition. It is thus possible to move the microorganisms between an initial nutrient medium and another nutrient medium different from the initial nutrient medium. This allows the ability of the microorganisms to develop in another nutrient medium to be evaluated, as described below.

As indicated above, the method allows a large field of observation to be addressed. Specifically, it is possible to place the carrier at a small distance from the image sensor 20, for example at a distance d smaller than 1 cm, or even to place it in contact with the image sensor. A large field of observation, the size of which is essentially dependent on the size of the image sensor, is thus achieved. Placing the image sensor at a sufficiently small distance d from the carrier makes it possible to avoid the need to use an image-forming optical system between the image sensor 20 and the carrier 15.

Apart from good transmittance properties in the infrared, the material from which the carrier 15 is formed is preferably not cytotoxic to the characterized microorganisms. It is preferably sterilizable, and compatible with conventional sterilization methods: sterilization by exposure to ionizing radiation (gamma radiation or x-rays for example) or non-ionizing radiation (microwaves or ultraviolet radiation), chemical sterilization (strong oxidants) or thermal sterilization (wet heat, dry heat, autoclave).

The formation of pores 15₃ in the material from which the carrier is formed may result from the application of a chemical method, electrochemical oxidation for example, or of a physical method, exposure to an electron beam or a beam of heavy ions for example. The method used to form the pores must allow through-pores 15₃ (i.e. pores that extend from the first face 15₁ to the second face 15₂) that are preferably of controlled size to be formed.

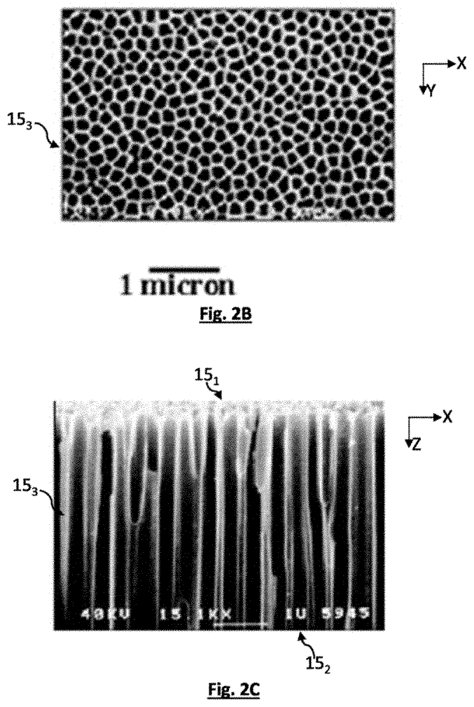

FIG. 2A shows one example of a spectrum of the absorbance of alumina (curve a), for a wavelength spectrum extending between 5.6 μm and 8 μm (wave number comprised between 1200 cm⁻¹ and 1800 cm⁻¹). The absorbance is equal to 1 minus the transmittance. In FIG. 2A, the y-axis corresponds to absorbance (comprised between 0 and 1). The x-axes correspond to wave number (bottom axis—units cm⁻¹) and to wavelength (top axis—units microns μm).

FIG. 2A also shows the absorbance spectra of various microorganisms: Candida albicans (curve a1), Escherichia coli (curve a2), Listeria inoccua (curve a3), Enterobacter cloacae (curve a4). The absorption peaks of various chemical bonds have also been represented by dotted lines. These peaks have been collated in table 1.

TABLE 1

| Reference | Wave number (cm⁻¹) | Wavelength (μm) | Chemical bond |
|---|---|---|---|
| b₁ | 1235 | 8.1 | Phosphodiester |
| b₂ | 1400 | 7.14 | Carboxylate |
| b₃ | 1468 | 6.83 | Methylene |
| b₄ | 1535 | 6.67 | Amide II |
| b₅ | 1655 | 6.05 | Amide I |
| b₆ | 1715 | 5.8 | Carboxylic acids |
| b₇ | 1738 | 5.74 | Esters |

Two reference wavelengths (b₈ and b₉) have also been shown. The latter correspond to wavelengths that are considered not to be significantly absorbed by bacteria. Images formed at these wavelengths are representative of the absorbances of the carrier 15, and of the nutrient medium present in the pores 15₃, and of any gases present between the carrier and the image sensor, in particular CO₂ or water vapor.

In FIG. 2A, it may be seen that, in a spectral band extending between 5.6 μm and 8 μm, the absorbance of alumina is comprised between 0.98 and 0.2, this corresponding to a transmittance comprised between 0.02 and 0.8.

FIG. 2B is an image, taken by scanning electron microscope, of one face of an alumina carrier obtained by anodic oxidation of an aluminum sheet. Such a material is conventionally designated porous anodic aluminum oxide.

The pores, the size of which is distributed about an average value of 200 nm, may be seen to have a regular arrangement. The distribution of the size of the pores is considered to be uniform. In this example, porosity (volume fraction of the pores), measured by image processing, was estimated to be 59%. The higher the porosity, the higher the amount of potentially absorbent nutrient medium in the pores. The lower the porosity, the more difficult it is for the nutrient medium to reach the microorganisms. Generally, the porosity may be comprised between 0.1% and a few tens of %, for example 70% or 80%.

FIG. 2C is a cross-sectional view through the thickness of the carrier shown in FIG. 2B. In this example, the thickness of the carrier 15 is 60 μm. It may be seen that the pores are through-pores, and extend from the first face 15₁ of the carrier to the second face 15₂ of the carrier.

The inventors have devised a method for observing microorganisms, and more precisely colonies of microorganisms, the main steps of which are shown in FIG. 3 and described below.

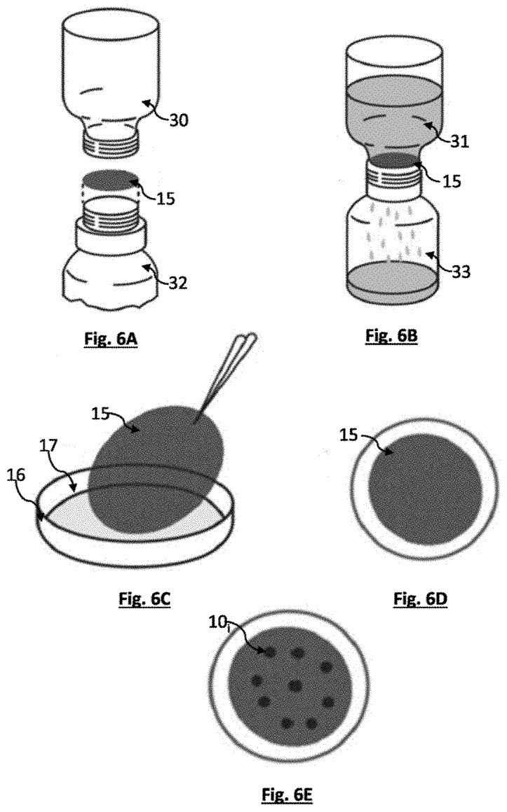

Step 100: placing microorganisms 10ᵢ on the first face 15₁ of the porous carrier 15. This step may consist in seeding the microorganisms using a spreading method, or in one of the variants described with reference to FIGS. 6A and 6B.

Step 110: placing the porous carrier 15 in contact with a nutrient medium 17, such that the microorganisms may develop. Preferably, in this step, the second face 15₂ of the porous carrier 15 is placed on the free surface of the nutrient medium. In this step, the nutrient medium is contained in a chamber 16, a petri dish for example. As described above, the nutrient medium 17 diffuses through the pores 15₃ so as to reach the microorganisms 10ᵢ placed on the first face 15₁. This allows colonies to develop.

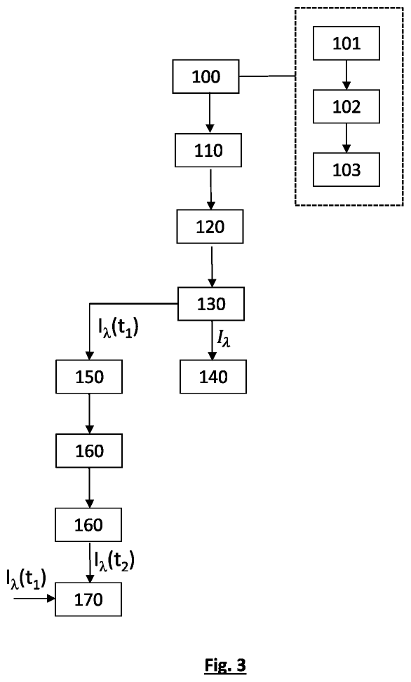

FIG. 4A shows a chamber 16 comprising an agar nutrient medium, on which Enterobacter cloacae bacteria are developing, so as to form colonies. A porous carrier has been placed on this TSA nutrient medium (TSA standing for tryptic soy agar). In FIG. 4A, the porous carrier 15 has been framed by a dotted circle. It may be seen that the bacterial colonies develop comparatively on the porous carrier and on the nutrient medium beyond the porous carrier. This shows that the bacteria are able to develop on the porous carrier 15.

FIGS. 4B and 4C are images, acquired over time, of Enterobacter cloacae bacteria developing on an LB agar nutrient medium (LB standing for Luria Bertani or lysogeny broth). In FIG. 48, the microorganisms developed directly on the surface of the nutrient medium. In FIG. 4C, the microorganisms developed on the surface of a porous alumina carrier the pores of which had a diameter of 200 nm. The porous alumina carrier was placed on the nutrient medium. These figures confirm that the development of the microorganisms, on the porous carrier, is comparable to their development directly on the nutrient medium. In other words, the presence of the porous carrier does not affect, or if so negligibly, the development of the microorganisms.

Step 120: removing the porous carrier 15 from the nutrient medium 17, and moving the porous carrier relative to the chamber 16. In this step, the porous carrier 15 is interposed between a light source 11 and an image sensor 20, such as described with reference to FIG. 1C. Preferably, the first face 15₁ is placed facing the light source 11 and the second face 15₁ is placed facing the image sensor 20, however this is not essential. Provision may be made to place the first face 15₁ facing the image sensor 20 and the second face 15₂ facing the light source 11.

Step 130: illuminating the carrier 15 at at least one wavelength λ, in the infrared domain, as described with reference to FIG. 1C, and acquiring an image I_{λ}. The acquired image allows colonies present on the carrier 15 to be observed. Preferably, step 130 is reiterated at various wavelengths λ, in the infrared domain. During each illumination, the light source 11 emits an incident light wave 12 the spectral band of which is preferably narrow, as already described. Thus, as many images I_{λ} are obtained as there are successive illumination wavelengths. If N wavelengths λ₁ . . . λ_{N} are employed, N images I_{λ1} . . . I_{λN}, forming a stack of images, are thus obtained.

Step 140: characterization

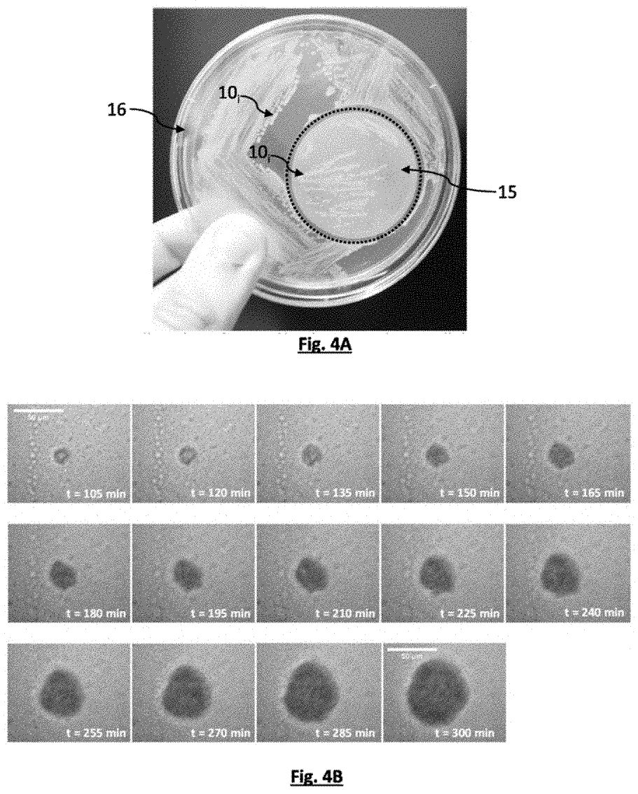

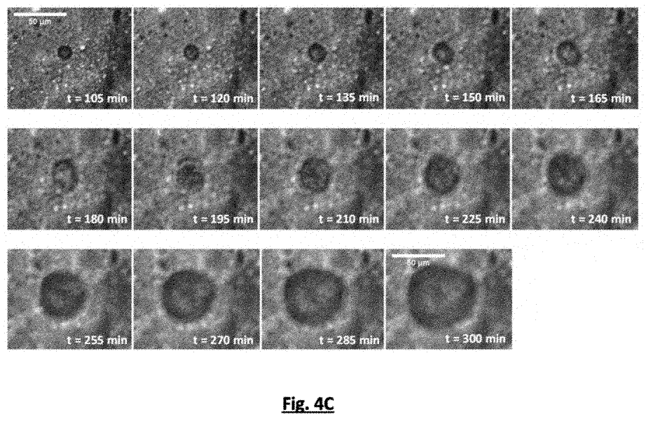

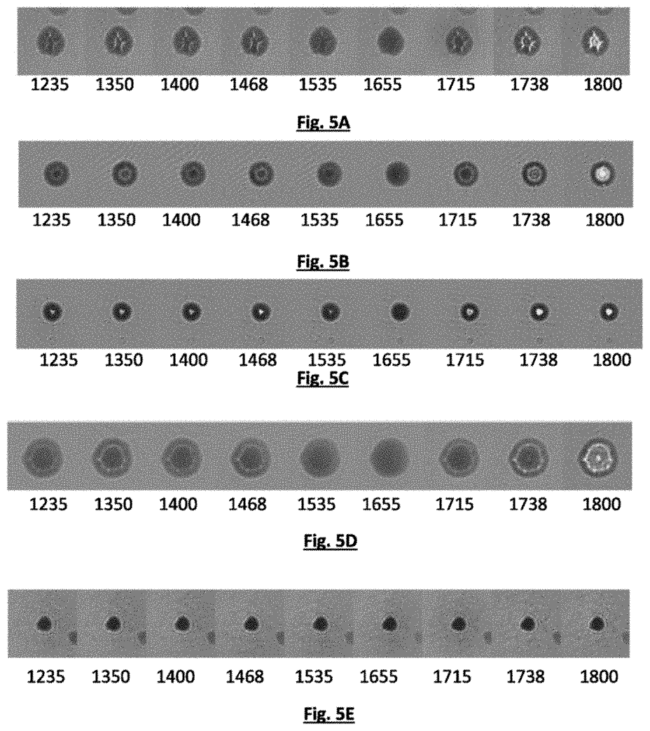

From the images of the stack of images I_{λ1} . . . I_{λN}, it is possible to extract thumbnails I_{i,λ1} . . . I_{i,λN}, each thumbnail corresponding to a given colony observed in each image. Each thumbnail is a region of interest of an image, corresponding to a given colony. Examples of images, corresponding to various bacterial species will be described with reference to FIGS. 5A to 5E, respectively.

Each thumbnail I_{i,λ} corresponds to a signature of the colony of microorganisms 10ᵢ at the wavelength I_{i,λ}. The signature depends:
on chemical composition, because the brightness of each thumbnail I_{i,λ} depends on the absorption, by the examined colony of microorganisms 10ᵢ, at the wavelength λ.
on the morphology of the colony (morphotype), which depends on the species of the microorganisms. Morphology may be characterized by morphological indicators, for example moments of order 3 or 4, usually designated skewness or kurtosis. The morphotype may also be characterized by Zernike or Fourier-Bessel moments, these moments being known in the field of image classification. Morphotype may also be characterized by image-texture indicators, and for example by a Haralick matrix.

The thumbnails I_{i,λ} allow the bacteria 10 to be identified. Characterization of the thumbnails thus allows the bacterial species forming the colony to be identified.

Step 140 may comprise identifying each colony on the basis of the thumbnails I_{i,λ} corresponding to said colony. To this end, the images are processed by a classifying algorithm, which is implemented by the processing unit 21. The processing algorithm may for example be an artificial-intelligence algorithm and for example a supervised-learning algorithm. It may for example be a SVM algorithm (SVM standing for support vector machine) or a neural-network algorithm or a random-forest algorithm. Use of a supervised-learning algorithm assumes a prior phase of training using images corresponding to known species of microorganisms.

### Experimental Trials

Steps 100 to 140 were implemented using a carrier consisting of a membrane made of porous aluminum oxide: reference Anodisc (registered trademark)—manufacturer Whatman. The thickness of the carrier was 80 μm. The diameter of the pores was 200 nm. The carrier was sterilized by autoclave (pressure of 1 bar steam at 121.1° C. for 15 minutes). The carrier was placed on a solid nutrient medium, at 37° C., for an incubation time of 24 h. After incubation, the carrier was deposited directly on an image sensor, the bolometers of this image sensor being of 25 μm side length and of 37 μm pitch. The light source was a laser source tunable in steps of 1 cm⁻¹, reference MIRcat—manufacturer Daylight Solutions, allowing an incident light wave of wavelength comprised between 5 μm and 11 μm to be emitted.

FIGS. 5A to 5E show thumbnails I_{i,λ1} . . . I_{i,λN} extracted from a stack of acquired images I_{λ1} . . . I_{λN} and corresponding to bacteria of the following types Enterobacter cloacae, Escherichia coli, Candida albicans, Staphylococcus epidermis, Listeria innocua, respectively. The thumbnails were obtained by successively illuminating each colony at 10 wavelengths comprised between 1235 cm⁻¹ and 1800 cm⁻¹. The wave number corresponding to each illumination has been indicated below each thumbnail. Such thumbnails were used to train an SMO SVM algorithm (SMO standing for sequential minimal optimization). The input data of the algorithm were descriptors of each thumbnail, and especially moments of order 3 (skewness) or of order 4 (kurtosis). The output data were the class to which the corresponding input data belonged. Each species in question was associated with one class.

The inventors acquired thumbnails of 1012 colonies. The database thus formed was partitioned into 10 groups of equal size with a view to cross-validation. For each partition, 90% of the thumbnails (9 groups) were used for the purposes of supervised learning and 10% of the thumbnails (1 group) was used to test the performance of the classifying algorithm. Table 2 collates, in a confusion matrix, the percentage (between 0 and 1) of correct classifications in the test phases.

TABLE 2

| | Candida | Escherichia | Enterobacter | Listeria | Staphyloccocus |
|---|---|---|---|---|---|
| | albicans | coli | cloacae | innocua | epidermis |
| Candida | 0.988 | 0.00 | 0.004 | 0.00 | 0.008 |
| albicans | | | | | |
| Escherichia coli | 0.0 | 0.994 | 0.00 | 0.00 | 0.006 |
| Enterobacter | 0.0 | 0.018 | 0.993 | 0.012 | 0.037 |
| cloacae | | | | | |
| Listeria | 0.0 | 0.007 | 0.0 | 0.954 | 0.039 |
| innocua | | | | | |
| Staphyloccocus | 0.004 | 0.019 | 0.011 | 0.026 | 0.94 |
| epidermis | | | | | |

Table 2 shows that the various species of microorganisms may be identified with a satisfactory confidence level. This attests to the relevance of the invention.

### Variants

According to a first variant, the porous carrier 15 is also used as a filter, so as to retain microorganisms in a fluid medium, prior to their identification. This variant is illustrated in FIGS. 6A to 8F. It corresponds to sub-steps 101 to 103 of step 100 described above.

Sub-step 101: mounting of the carrier. FIG. 6A shows an upstream reservoir 30 and a downstream reservoir 32. The porous carrier 15 is placed between the upstream reservoir 30 and the downstream reservoir 32. The upstream reservoir is intended to receive a fluid 31 to be analyzed, liable to contain microorganisms. In this example, the fluid is a liquid.

Sub-step 102: filtration. This step is shown in FIG. 6B. The upstream reservoir 30 is filed with the fluid 31 to be analyzed. The latter flows through the porous carrier 15. Any microorganisms 10ᵢ present in the fluid are retained by the filter. This step allows the microorganisms initially present in the upstream reservoir 30, which may have a large volume, 100 mL for example, to be concentrated on the porous carrier 15. The filtered fluid 33 may be collected in the downstream reservoir 32.

Sub-step 103: removal of the filter: the porous carrier 15 is removed so as to be placed on the surface of a nutrient medium 17.

Afterwards, the porous carrier 15 is deposited on the surface of the nutrient medium 17, the nutrient medium occupying a chamber 16 (see FIG. 6C). FIGS. 6D and 6E schematically show the development of colonies of microorganisms on the porous carrier in contact with the nutrient medium, this corresponding to step 110 described above. Steps 120 to 140 may then be implemented, so as to identify the species of microorganisms and/or to count the number of colonies formed, this optionally being done for each identified species. The number of colonies may be divided by the volume (or mass) of fluid 31, this allowing a microbial load per unit mass or volume to be computed (in colony-forming units (cfu) per gram or per milliliter for example).

The inventors have implemented the steps schematically shown in FIGS. 6A to 6E using a porous alumina carrier, the average size of the pores of which was 200 nm. The porous carrier was placed on a chromogenic nutrient medium. Such a medium has the property of coloring the developing microorganisms. FIG. 6F is a photograph of a porous carrier that was left in contact with a chromogenic nutrient medium for an incubation time of 24 hours. The chromogenic nutrient medium was a Chromid (registered trademark) CPS (registered trademark) chromogenic agar—manufacturer Biomérieux. The presence of dark spots, which correspond to colonies of bacteria (Escherichia coli) colored following their development in the chromogenic nutrient medium, will be observed.

An example of application of the first variant is to bacteriological analysis of water intended for human consumption. Alternatively, the fluid 31 is a gas. The invention allows microorganisms carried in a gas, air for example (industrial air, air in a hospital environment), to be retained and analyzed.

According to a second variant, the characterization of the microorganisms consists not in identifying them, but in evaluating their ability to develop. This allows indicators of sensitivity to a bactericide or a bacteriostat, an antibiotic for example, to be obtained. One example of an indicator is minimum inhibitory concentration (MIC), which corresponds to the minimum antibiotic concentration at which development of bacteria is inhibited.

Thus, the nutrient medium may contain an antibiotic with a known concentration, or with a known spatial concentration gradient, and for example a high concentration at its center and a low concentration on its periphery. Images of the carrier 15 may be taken at an initial time t and at a second time t₂, respectively. Between the times t₁ and t₂, the carrier is placed on the surface of a nutrient medium containing an antibiotic. Comparison of the images obtained at the initial time t₁ and at the second time t₂, respectively, allows the development of the colonies between the two times to be evaluated. It is thus possible to characterize the ability of the analyzed microorganism to develop in the presence of the antibiotic.

According to one possibility, between the times t₁ and t₂, the nutrient medium contains, in addition to an antibiotic, an isotopic label present in a concentration higher than its natural concentration. The isotopic label is a molecule containing a stable isotope of an element. It may for example be a question of deuterium (D), substituted for hydrogen (H). Such a label is obtained using heavy water (D₂O). The labelled nutrient medium may for example be a Mueller-Hinton medium containing heavy water, and a known concentration (or a known spatial concentration gradient). At the second time, the porous carrier 15 is illuminated at a wavelength absorbed by a chemical bond involving the isotope. It may for example be a question of the carbon-deuterium bond when the isotope is deuterium. If such a bond is revealed, by an image, to be present, it may be concluded that metabolism is ongoing in the labelled nutrient medium. Conversely, if the absence of such a bond is revealed it may be concluded that the bacteria is not metabolizing.

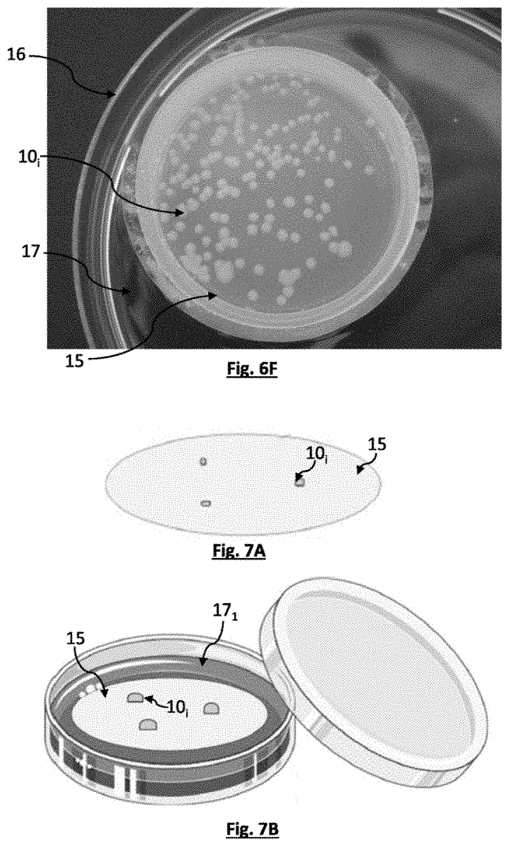

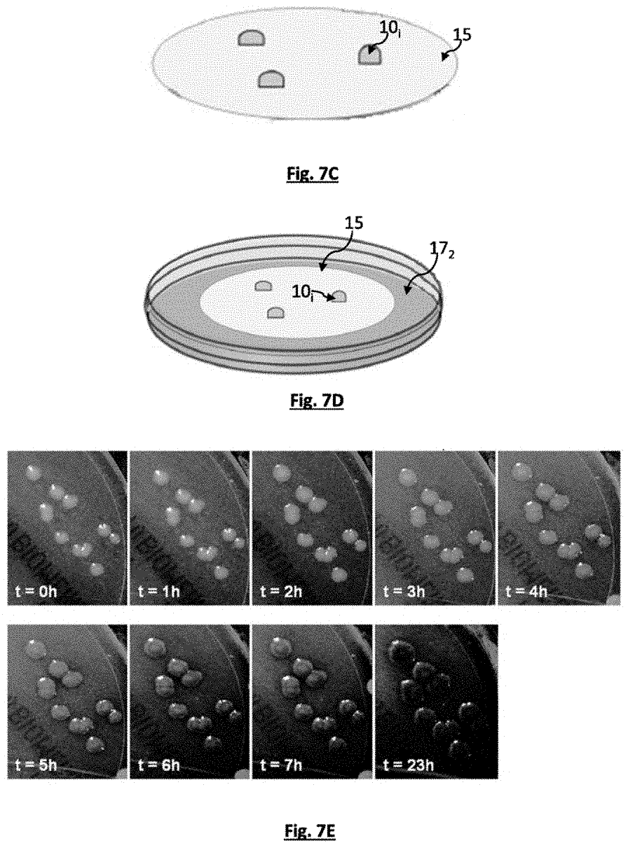

This variant corresponds to steps 100 to 130 and 150 to 170 shown in FIG. 3, and to FIGS. 7A to 7E. Steps 100 to 130 are implemented as described above. FIGS. 7A and 7B illustrate steps 100 (seeding) and 110 (incubation). In step 110, the nutrient medium used is an initial medium 17₁, containing no antibiotics. In step 120, the porous carrier 15 is removed from the initial nutrient medium (see FIG. 7C). Step 130 is implemented at an initial time t₁, using a device such as shown in FIG. 1C. This leads to the obtainment of initial images I_{λ}(t₁).

In step 150, the porous carrier is placed in contact with a second nutrient medium 17₂ (see FIG. 7D). The second nutrient medium 17₂ contains a known antibiotic concentration, or a known spatial antibiotic concentration gradient. The second medium 17₂ may contain an isotopic label, for example as a result of the addition of heavy water.

In step 160, the porous carrier is removed from the second nutrient medium 17₂, then placed in an observing device such as shown in FIG. 1C.

In step 170, second images I_{λ}(t₂) are formed at a second time t₂ subsequent to the initial time t₁. Comparison of the initial images I_{λ}(t₁) and second images I_{λ}(t₂) allows the ability of the microorganisms to metabolize in the second medium to be evaluated.

Instead of an isotopic label, a label containing a known covalent bond having an exploitable absorbance at an illumination wavelength of the light source may be used. It may for example be a question of the C≡N bond, which has an absorption wavelength at 2200 cm⁻¹. Formation of images at said wavelength allows the metabolic activity of the microorganism in the second medium to be evaluated.

Instead of an isotopic label, a chromogenic medium able to induce a modification of the color of the microorganisms under the effect of their metabolism may be used. FIG. 7E illustrates such an alternative. This figure shows a variation in the color of colonies of Escherichia coli. These colonies were initially cultured on a porous carrier resting on an initial nutrient medium (TSA—Tryptic Soy Agar). The porous carrier was then deposited on a Chromid (registered trademark) CPS (registered trademark) chromogenic medium—manufacturer Biomérieux. The bacteria were observed to gradually change color, from white to red.

The invention allows a high number of microorganisms to be characterized simultaneously and non-destructively. It does not require complex instrumentation and is particularly simple to implement. Furthermore, it is easily automatable.

1. A method for characterizing microorganisms, the method comprising:
(a) depositing microorganisms on a porous carrier, the porous carrier comprising a first face and a second face, and pores extending from the first face to the second face, the microorganisms being retained on the first face;
(b) placing the porous carrier on the surface of a nutrient medium contained in a chamber, the porous carrier being placed such that the second face is placed in contact with the nutrient medium, so that the nutrient medium diffuses from the second face to the first face, through the pores;
(c) moving the porous carrier with respect to the chamber;
(d) positioning the porous carrier between an infrared light source and an image sensor, the light source being configured to emit an incident light wave at an emission wavelength, the porous carrier transmitting all or some of the incident light wave at the emission wavelength;
(e) illuminating the microorganisms, placed on the porous carrier, with the light source and acquiring an image with the image sensor, at the emission wavelength, the image allowing at least one colony of microorganisms to be observed;
(f) characterizing the colony of microorganisms on the basis of the image acquired in step e); and
(g) repeating the illuminating (e), the microorganisms being successively illuminated at various emission wavelengths, so as to obtain as many images as there are emission wavelengths;
wherein the characterizing (f) is performed based on the images acquired in the repeating (g).

2. The method of claim 1, wherein the characterizing (f) comprises:
identification of the species of the microorganisms forming the colony; or
a determination of the ability of the microorganisms to develop in the nutrient medium.

3. The method of claim 1, wherein the porous carrier transmits a least 1% of the light at the emission wavelength.

4. The method of claim 1, wherein the characterizing (f) comprises extracting a thumbnail from each image acquired in the illuminating (e),
wherein the thumbnail is a region of interest of the acquired image corresponding to the colony of microorganisms.

5. The method of claim 4, wherein, in the characterizing (f), each thumbnail forms an input datum of a supervised-learning algorithm, with a view to characterizing the microorganisms forming a colony.

6. The method of claim 5, wherein the input data of the supervised-learning algorithm comprise morphological indicators of each thumbnail.

7. The method of claim 6, wherein the morphological indicators comprise moments of order 3 and/or of order 4 computed on each thumbnail.

8. The method of claim 1, wherein the depositing (a) comprises seeding the porous carrier.

9. The method of claim 1, wherein the depositing (a) comprises making a fluid, suitable to comprise microorganisms, flow through the porous carrier, from the first face to the second face, the carrier acting as a filter, so as to retain microorganisms on the first face, while permitting the fluid to flow through the pores.

10. The method of claim 1, wherein, in the placing (b), the nutrient medium:
comprises molecules labelled with an isotope; or
is a chromogenic substrate;
comprises molecules having a chemical bond that absorbs light at the emission wavelength
such that the image formed in the illuminating (e), or each image formed in the illuminating (e), is representative of a metabolic activity of the microorganisms in contact with the nutrient medium.

11. The method of claim 1, wherein, in the placing (b), the nutrient medium comprises a bactericide or a bacteriostat, such that the image formed in the illuminating (e), or each image formed in the illuminating (e), is representative of a metabolic activity of the microorganisms when the microorganisms are in contact with the nutrient medium.

12. The method of claim 1, comprising, following the depositing (a) and prior to the placing b), acquiring an initial image of the porous carrier, such that metabolic activity may be determined via a comparison between the initial image and at least one image acquired in the illuminating (e).

13. The method of claim 1, wherein the thickness of the porous carrier, between the first face and the second face, is smaller than 1 mm.

14. The method of claim 1, wherein the diameter or largest diagonal of each pore is in a range of from 5 nm to 5 μm.

15. The method of claim 1, wherein the porous carrier is comprises alumina, silicon, germanium, zinc sulfide, silicon nitride, zinc selenide, a chalcogenide glass, calcium fluoride, or potassium bromide.

16. The method of claim 1, wherein the characterizing (f) is an identification of the species of the microorganisms forming the colony.

17. The method of claim 1, wherein the characterizing (f) is a determination of the ability of the microorganisms to develop in the nutrient medium.

18. The method of claim 1, wherein the porous carrier transmits a least 1% of the light at each emission wavelength.
